(19) United States
(12) Patent Application Publication
(10) Pub. No.: US 2020/0094025 A1
(43) Pub. Date: Mar. 26, 2020

(54) PERCUTANEOUS VASCULATURE ACCESS DEVICE

(71) Applicant: Medtronic Vascular, Inc., Santa Rosa, CA (US)

(72) Inventor: Elise L. Wisman, Santa Rosa, CA (US)

(21) Appl. No.: 16/581,090

(22) Filed: Sep. 24, 2019

Related U.S. Application Data
(60) Provisional application No. 62/736,903, filed on Sep. 26, 2018.

(51) Int. Cl. A61M 25/06; A61M 29/02; A61M 25/09; A61M 25/00; A61M 25/02

(52) U.S. Cl. CPC A61M 25/0625; A61M 29/02; A61M 25/0662; A61M 25/09041; A61M 2025/024; A61M 25/02; A61M 2025/0098; A61M 2025/0253; A61M 25/0097

(57) ABSTRACT

A vasculature access device may include a needle, a dilator, an introducer sheath, a hub, and manipulators in a single, integrated device. The vasculature access device may also optionally include one or both of a guidewire and one or more features for facilitating restraint of the hub and the introducer sheath relative to a patient. By integrating the needle, dilator, introducer sheath, hub, and manipulators in a single device, exchanges of these components (e.g., removing one component from the vasculature and introducing a different component into the vasculature), which is done in access kits where at least some of these components are separate, are reduced.

### CROSS-REFERENCE TO RELATED APPLICATIONS

This application claims the benefit of U.S. Application Ser. No. 62/736,903, filed Sep. 26, 2018, the disclosure of which is hereby incorporated by reference in its entirety.

### TECHNICAL FIELD

This disclosure relates to medical devices for percutaneously accessing vasculature of a patient.

### BACKGROUND

Percutaneous accessing of vasculature is used for many medical interventions. For example, to perform a percutaneous coronary intervention, a clinician may gain access to coronary vasculature from the femoral artery via Seldinger's technique.

### SUMMARY

This disclosure describes vasculature access devices that include a needle, a dilator, an introducer sheath, a hub, and manipulators in a single, integrated device. The vasculature access devices may also optionally include one or both of a guidewire and one or more features for facilitating restraint of the hub and the introducer sheath relative to a patient. By integrating the needle, dilator, introducer sheath, hub, and manipulators in a single device, exchanges of these components (e.g., removing one component from the vasculature and introducing a different component into the vasculature), which is done in access kits where at least some of these components are separate, are reduced. This may increase speed and efficiency in completing a vasculature access procedure, reduce a risk of complications, including contamination of the components, patient infection, reduce a possibility of compromising the integrity of one of the components, or the like.

In at least one embodiment, a percutaneous vasculature access device is provided that includes a hub defining a proximal hub end, a distal hub end, and a hub lumen extending from the proximal hub end to the distal hub end. An introducer sheath may define a proximal introducer sheath end, a distal introducer sheath end, and an introducer sheath lumen extending from the proximal introducer sheath end to the distal introducer sheath end. The proximal introducer sheath end may be configured to be connected to the distal hub end, and the introducer sheath lumen may be in communication with the hub lumen. A dilator may define a dilator lumen and a proximal attachment may be configured to be connected to the dilator. The proximal attachment may be configured to be removably connected to the proximal hub end, and the dilator may be configured to extend distally from the distal introducer sheath end when the proximal attachment is configured to be removably connected to the proximal hub end. A needle may be configured to be movably disposed within the dilator lumen and a needle cap may be configured to be removably connected to a proximal end of the proximal attachment. The needle cap may be configured to be connected to the needle such that movement of the needle cap causes movement of the needle.

In one embodiment, the the needle defines a needle lumen and the device further comprises a guidewire configured to be advanced into the needle lumen.

The percutaneous vasculature access device may further include a guidewire actuator configured to be connected to the guidewire and configured to cause the guidewire to move longitudinally relative to the needle.

In one embodiment, the proximal attachment defines a longitudinal slot, the device further comprising a guidewire actuator configured to be connected to the guidewire and configured to move longitudinally in the longitudinal slot relative to the proximal attachment to move the guidewire relative to the needle.

The guidewire actuator may be configured to be connected to the guidewire so that movement of the guidewire actuator causes a corresponding movement of the guidewire.

In one embodiment, the guidewire actuator comprises a ridged tab.

Proximal removal of the proximal attachment from the hub proximal end may be configured to cause proximal removal of the guidewire and the dilator out of the hub lumen.

The dilator may be visually translucent or visually transparent.

The percutaneous vasculature access device may include a needle hook configured to connect the needle to the needle cap.

The needle hook may include at least one aperture fluidically coupled to the needle lumen.

In one embodiment, the proximal attachment defines a proximal attachment lumen extending from the proximal end of the proximal attachment to a distal end of the proximal attachment, and the needle hook extends from the needle to the needle cap through the proximal attachment lumen.

Proximal removal of the needle cap may be configured to cause proximal removal of the needle out of the hub lumen.

The hub may include side attachments configured to receive a band or tape to secure the hub and the introducer sheath to a patient.

The hub may include at least one clip feature configured to engage with a hub band clip, wherein the hub band clip is configured to secure the hub and the introducer sheath to a patient.

The hub may include a side access port.

Proximal removal of the proximal attachment from the hub proximal end may be configured to be proximal removal of the dilator out of the hub lumen.

In at least one embodiment, a percutaneous vasculature access device is provided including a hub defining a proximal hub end, a distal hub end, and a hub lumen extending from the proximal hub end to the distal hub end. An introducer sheath may extend from the distal hub end, wherein the introducer sheath defines a proximal introducer sheath end, a distal introducer sheath end, and an introducer sheath lumen extending from the proximal introducer sheath end to the distal introducer sheath end. The proximal introducer sheath end may be configured to be connected to the distal hub end, and the introducer sheath lumen may be in communication with the hub lumen. A dilator may define a dilator lumen and a proximal attachment may be configured to be connected to the dilator. The proximal attachment may be configured to be removably connected to the proximal hub end, and the dilator may be configured to extend distally from the distal introducer sheath end when the proximal attachment is configured to be removably connected to the proximal hub end. A needle may be configured to be movably disposed within dilator lumen, wherein the needle defines a needle lumen. A needle cap may be configured to be removably connected to a proximal end of the proximal attachment, wherein the needle cap is configured to be connected to the needle such that movement of the needle cap causes movement of the needle. Proximal removal of the needle cap from the proximal attachment may be configured to cause proximal removal of the needle out of the hub lumen. A guidewire may be configured to be movably disposed in the needle lumen and a guidewire actuator may be configured to be connected to the guidewire. Movement of the guidewire actuator may be configured to cause movement of the guidewire relative to the needle, and proximal removal of the proximal attachment from the hub proximal end may be configured to cause proximal removal of the dilator and the guidewire out of the hub lumen.

In one embodiment, the proximal attachment defines a longitudinal slot, wherein the guidewire actuator is configured to move longitudinally in the longitudinal slot relative to the proximal attachment to move the guidewire longitudinally relative to the needle.

The guidewire actuator may be configured to connected to the guidewire so that movement of the guidewire actuator causes a corresponding movement of the guidewire.

The guidewire actuator may include a thumb tab.

The dilator may be visually translucent or visually transparent.

The percutaneous vasculature access device may include a needle hook configured to connect the needle to the needle cap.

The needle hook may include at least one aperture fluidically coupled to the needle lumen.

In one embodiment, the proximal attachment defines a proximal attachment lumen extending from the proximal end of the proximal attachment to a distal end of the proximal attachment, and the needle hook is configured to extend from the needle to the needle cap through the proximal attachment lumen.

The hub may include side attachments configured to receive a band or tape configured to secure the hub and the introducer sheath to a patient.

The hub may include at least one clip feature configured to engage with a hub band clip, wherein the hub band clip is configured to secure the hub and the introducer sheath to a patient.

The hub may include a side access port.

In at least one embodiment, a method is provided including advancing a percutaneous vasculature access device relative to anatomy of a patient to cause a needle of the percutaneous vasculature access device to be inserted percutaneously into vasculature of a patient. The percutaneous vasculature access may include: a hub defining a proximal hub end, a distal hub end, and a hub lumen extending from the proximal hub end to the distal hub end; an introducer sheath defining a proximal introducer sheath end, a distal introducer sheath end, and an introducer sheath lumen extending form the proximal introducer sheath end to the distal introducer sheath end, wherein the proximal introducer sheath end is configured to be connected to the distal hub end, and wherein the introducer sheath lumen is in communication with the hub lumen; a dilator defining a dilator lumen; a proximal attachment configured to be connected to the dilator, wherein the proximal attachment is configured to be removably connected to the proximal hub end, and wherein the dilator configured to extend distally from the distal introducer sheath end when the proximal attachment is configured to be removably connected to the proximal hub end; the needle, which is configured to be movably disposed within dilator lumen; and a needle cap configured to be removably connected to a proximal end of the proximal attachment, wherein the needle cap is configured to be connected to the needle such that movement of the needle cap causes movement of the needle. The method may further include advancing the dilator, the introducer sheath, and the needle into the vasculature, removing the needle cap from the proximal end of the proximal attachment to cause removal of the needle from the vasculature and out of the proximal hub end of the hub lumen, and removing the proximal attachment from the proximal hub end to cause removal of the dilator from the vasculature and out of the proximal hub end of the hub lumen.

The method may further include securing the hub to an anatomical structure of the patient using a hub clip band.

In at least one embodiment, a method is provided including advancing a percutaneous vasculature access device relative to anatomy of a patient to cause a needle of the percutaneous vasculature access device to be inserted percutaneously into vasculature of a patient. The percutaneous vasculature access device may include: a hub defining a proximal hub end, a distal hub end, and a hub lumen extending from the proximal hub end to the distal hub end; an introducer sheath configured to extend from the distal hub end, wherein the introducer sheath defines a proximal introducer sheath end, a distal introducer sheath end, and an introducer sheath lumen extending from the proximal introducer sheath end to the distal introducer sheath end, and wherein the introducer sheath lumen is in communication with the hub lumen; a dilator defining a dilator lumen; a proximal attachment configured to be connected to the dilator, wherein the proximal attachment is configured to be removably connected to the proximal hub end, and wherein the dilator is configured to extend distally from the distal introducer sheath end; a needle configured to be movably disposed within dilator lumen, wherein the needed defines a needle lumen; a needle cap configured to be removably connected to a proximal end of the proximal attachment, wherein the needle cap is configured to be connected to the needle such that movement of the needle cap causes movement of the needle, wherein proximal removal of the needle cap from the proximal attachment is configured to cause proximal removal of the needle out of the hub lumen; a guidewire configured to be movably disposed in the needle lumen; and a guidewire actuator configured to be connected to the guidewire, wherein movement of the guidewire actuator is configured to cause movement of the guidewire relative to the needle, and wherein proximal removal of the proximal attachment from the hub proximal end is configured to cause proximal removal of the dilator and the guidewire out of the hub lumen. The method may further include actuating the guidewire actuator to advance the guidewire out of a distal end of the needle lumen, removing the needle cap from the proximal end of the proximal attachment to cause removal of the needle from the vasculature and out of the proximal hub end of the hub lumen, and advancing the percutaneous vasculature access device to advance the guidewire through the vasculature and advance the dilator and introducer sheath into the vasculature. The proximal attachment may be removed from the proximal hub end to cause removal of guidewire and the dilator from the vasculature and out of the proximal hub end of the hub lumen.

The method may further include securing the hub to the patient using a hub clip band.

The proximal attachment may define a longitudinal slot, and the method may include moving the guidewire actuator longitudinally in the longitudinal slot relative to the proximal attachment to move the guidewire longitudinally relative to the needle.

The guidewire actuator may be configured to be connected to the guidewire so that movement of the guidewire actuator causes a corresponding, substantially equal movement of the guidewire.

The guidewire actuator may include a thumb tab.

The details of one or more examples are set forth in the accompanying drawings and the description below. Other features, objects, and advantages will be apparent from the description and drawings, and from the claims. It is to be understood that embodiments disclosed herein may be combined to form additional embodiments.

### BRIEF DESCRIPTION OF DRAWINGS

FIG. 1 is a perspective view illustrating an example percutaneous vasculature access device, which includes a hub, an introducer sheath, a dilator, a needle, and manipulators.

FIG. 2 is a perspective view illustrating a distal portion of the percutaneous vasculature access device of FIG. 1, showing further details of the needle, the dilator, the introducer sheath, and a needle hook.

FIG. 3 is a perspective view illustrating a proximal portion of the percutaneous vasculature access device of FIG. 1, showing further details of the hub and manipulators that include a proximal attachment, a guidewire actuator, and a needle cap.

FIG. 4 is a flow diagram illustrating an example method for accessing vasculature using a percutaneous vasculature access device of this disclosure.

FIG. 5 is a perspective view illustrating the distal portion of the percutaneous vasculature access device of FIG. 1 after distal actuation of the guidewire actuator, showing a guidewire advanced out of a distal end of a needle lumen.

FIG. 6 is a perspective view illustrating the percutaneous vasculature access device of FIG. 1 with the needle cap removed from a proximal end of the proximal attachment.

FIG. 7 is a perspective view illustrating the hub and the introducer sheath of the percutaneous vasculature access device of FIG. 1, after removal of the proximal attachment, the dilator, the guidewire, the needle, and the needle cap.

FIG. 8 is a perspective view of an example introducer sheath and hub engaged with an example hub band clip.

FIG. 9 is a perspective view of an example introducer sheath and an example hub that includes side attachments configured to receive a band or tape.

FIG. 10 is a flow diagram illustrating another example method for accessing vasculature using a percutaneous vasculature access device of this disclosure.

### DETAILED DESCRIPTION

This disclosure describes vasculature access devices that include a needle, a dilator, an introducer sheath, a hub, and manipulators in a single, integrated device. The vasculature access devices may also optionally include one or both of a guidewire and one or more features for facilitating restraint of the hub and the introducer sheath relative to a patient. By integrating the needle, dilator, introducer sheath, hub, and manipulators in a single device, exchanges of these components (e.g., removing one component from the vasculature and introducing a different component into the vasculature), which is done in access kits where at least some of these components are separate, are reduced. This may increase speed and efficiency in completing a vasculature access procedure, reduce a risk of complications, including contamination of the components or patient infection, reduce a possibility of compromising the integrity of one of the components, or the like.

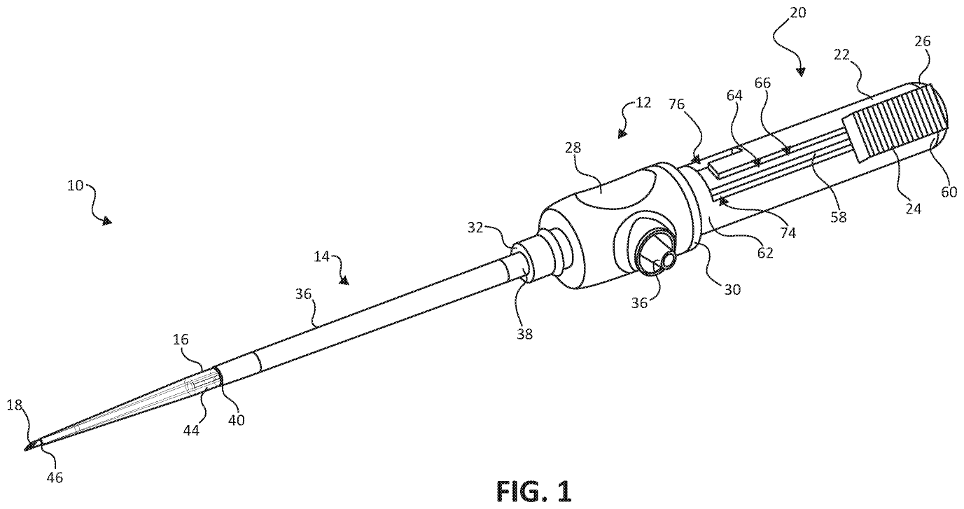

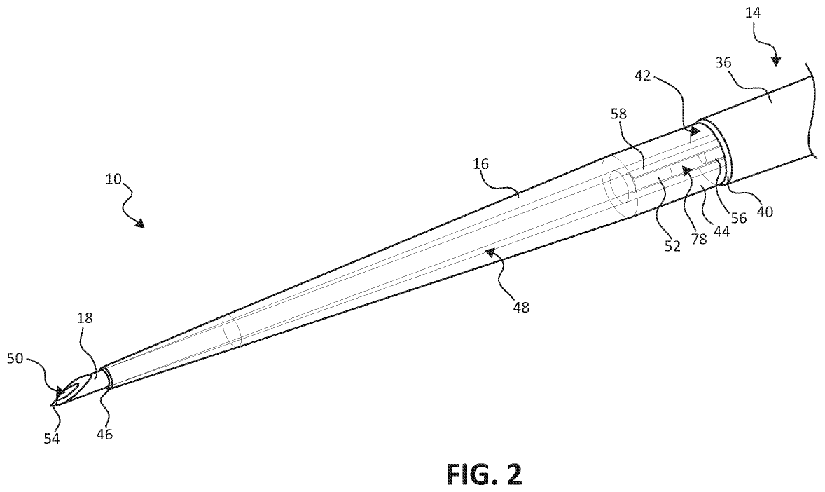

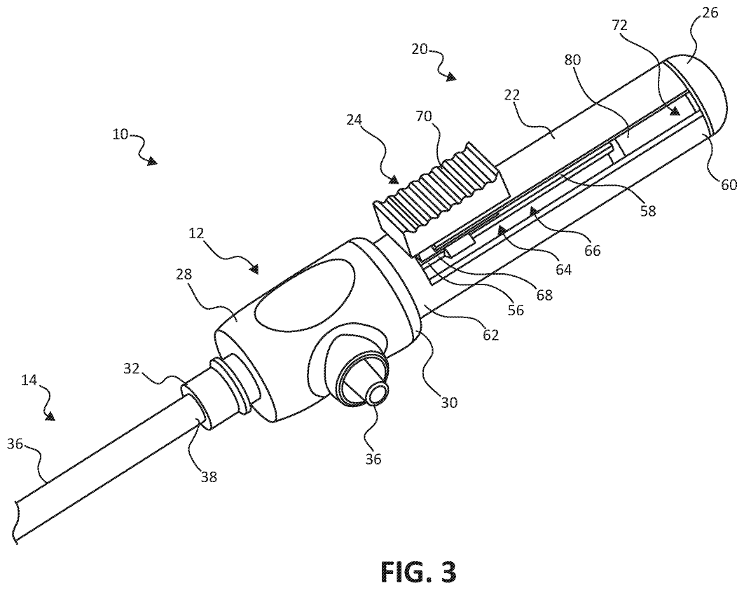

FIG. 1 is a perspective view illustrating an example percutaneous vasculature access device 10, which includes a hub 12, an introducer sheath 14, a dilator 16, a needle 18, and manipulators 20. FIG. 2 is a perspective view illustrating a distal portion of the percutaneous vasculature access device of FIG. 1, showing further details of needle 18, dilator 16, introducer sheath 14, and a needle hook 58. FIG. 3 is a perspective view illustrating a proximal portion of percutaneous vasculature access device 10 of FIG. 1, showing further details of hub 12 and a proximal attachment 22, a guidewire actuator 24, and a needle cap 26.

Manipulators 20 include a proximal attachment 22, a guidewire actuator 24, and a needle cap 26. Percutaneous vasculature access device 10 integrates hub 12, introducer sheath 14, dilator 16, needle 18, and manipulators 20 into a single device configured to percutaneously access vasculature of a patient and is used to introduce introducer sheath 14 into vasculature of the patient. Once introducer sheath 14 is introduced into vasculature of the patient, introducer sheath 14 and hub 12 provide an access point for introducing further medical devices, such as catheters, to the vasculature of the patient.

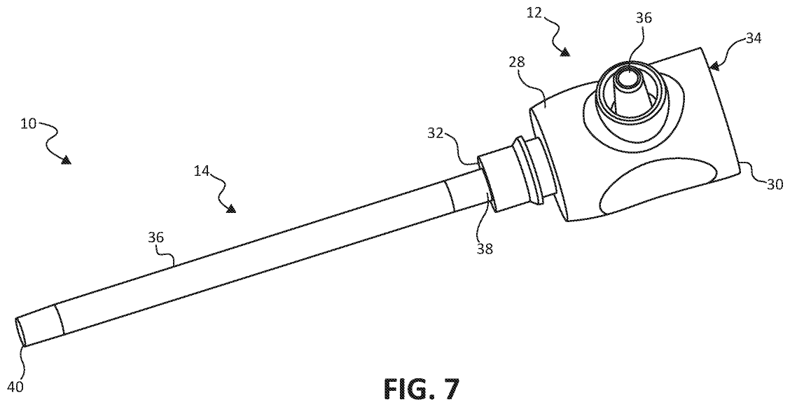

Hub 12 includes a hub body 28 that extends from a proximal hub end 30 to a distal hub end 32. Hub 12 provides an entrance structure for introduction of a medical device, such as a catheter, a balloon catheter, a prosthetic heart valve, a stent, or the like, into percutaneous vasculature access device 10, and, ultimately, into vasculature of a patient. In some examples, hub 12 may include one or more connection features, such as a leur lock, threads, or the like, for attaching an additional structure to proximal hub end 30 to facilitate introduction of the medical device to hub 12. Hub 12 also defines an internal hub lumen 34 (shown in FIG. 7) that extends from proximal hub end 30 to distal hub end 32. Hub lumen 34 may be sized and shaped to allow passage of a medical device, such as a catheter, a balloon catheter, a prosthetic heart valve, a stent, or the like through hub lumen 34.

In some examples, hub 12 also may optionally include a side port 36 that provides additional access to hub lumen 34. In some examples, side port 36 may open into hub lumen 34. In other examples, hub 12 may define two or more lumens and side port 36 may provide access to a different lumen than hub lumen 34. Side port 36 may be used to connect a side tube with stop cock to hub 12, such that a user can flush hub lumen 34, introducer sheath lumen 42, and the like with a sterile solution, such as saline.

Hub 12 may be formed form any suitable material. For example, hub 12 may be formed form a polymer, metal, or the like, and may be substantially rigid. In some examples, hub 12 is formed from a polycarbonate.

Introducer sheath 14 includes an introducer sheath body 36 that extends from a proximal introducer sheath end 38 to a distal introducer sheath end 40. Introducer sheath 14 functions to provide a lumen or channel though which a medical device passes from hub lumen 34 into vasculature of a patient. Introducer sheath 14 also defines an internal introducer sheath lumen 42 that extends from proximal introducer sheath end 38 to distal introducer sheath end 40. Introducer sheath lumen 42 is in communication with hub lumen 34. For example, introducer sheath lumen 42 may be coaxial or coincident with hub lumen 34, such that devices or components may pass easily from hub lumen 34 to introducer sheath lumen 42, and vice versa. In examples in which hub 12 includes multiple lumens, introducer sheath 14 may include a single lumen, or may include a respective, corresponding lumen for each lumen of hub 12.

Introducer sheath 14 is mechanically connected to hub 12. For example, a portion of introducer sheath 14 near proximal introducer sheath end 38 may be connected to a portion of hub 12 near distal hub end 32. In some examples, the connection may be substantially permanent, such that introducer sheath 14 is substantially non-removable from hub 12 without use of a chemical or tool (e.g., proximal introducer sheath end 38 may be adhered to distal hub end 32 using an adhesive, overmolded to distal hub end 32, or welded to distal hub end 32 using any suitable welding technique, such as ultrasonic welding). In other examples, proximal introducer sheath end 38 may be removably connected to distal hub end 32. For example, a portion of introducer sheath 14 near proximal introducer sheath end 38 may be engaged with a portion of distal hub end 32 in a friction fit or via a threaded connection. One or both of introducer sheath 14 or hub 12 may include engagement features that facilitate connection of introducer sheath 14 to hub 12, such as a tab and corresponding slot, mating protrusions and depressions, mating threads, or the like.

Introducer sheath body 36 may be formed from any suitable material. For example, introducer sheath body 36 may be formed from a biocompatible polymeric material. The biocompatible polymeric material may include, for example, a silicone, a fluoropolymer, a polyolefin, a fluorinated polyolefin, a polyurethane, a polyamide, a polyether, a polyether block amide, or the like. Introducer sheath 14 may include a single layer or multiple layers. For example, introducer sheath body 36 may include a first layer that provides structural properties to introducer sheath body 36 and a lubricious coating on an external surface of the first layer. In some examples, introducer sheath body 36 includes a first layer including fluorinated ethylene propylene and a coating including a silicon, a first layer including a polyether block amide and a coating including a hydrogel, or the like.

Introducer sheath 14 may define any suitable length, as measured from proximal introducer sheath end 38 to distal introducer sheath end 40. In some examples, introducer sheath 14 defines a length of about 7 cm, and 11 cm, or about 23 cm. In some examples in which percutaneous vasculature access device 10 may include a strain relief attached to distal hub end 32 fitting over a proximal portion of introducer sheath 14, the length of introducer sheath 14 may be measured from a distal end of the strain relief to distal introducer sheath end 40.

Although not shown in FIG. 1, in some examples, percutaneous vasculature access device 10 may include a strain relief attached to distal hub end 32 fitting over a proximal portion of introducer sheath 14. The strain relief may or may not be attached to introducer sheath 14. The strain relief may reduce a risk of introducer sheath 14 kinking or deforming as the interface between hub 12 and introducer sheath 14.

Dilator 16 extends distally from distal introducer sheath end 40. An exposed proximal dilator end 44 of dilator 16 is longitudinally adjacent distal introducer sheath end 40, and a distal dilator end 46 of dilator 16 is opposite exposed proximal dilator end 44. Dilator 16 defines a dilator lumen 48 extending a length of dilator 16 from the proximal-most end (not shown in FIG. 1 or 2) of dilator 16 to distal dilator end 46. Dilator lumen 48 is in communication with introducer sheath lumen 42 and hub lumen 34. For example, dilator lumen 48 may be coaxial with or coincident with introducer sheath lumen 42 and/or hub lumen 34.

Dilator 16 tapers in diameter from exposed proximal dilator end 44 to distal dilator end 46. In some examples, the taper begins at exposed proximal dilator end 44 and has a substantially continuous and consistent rate of taper to distal dilator end 46. In other examples, the taper may begin away from exposed proximal dilator end 44, end away from distal dilator end 46, or both. Similarly, the taper may be discontinuous (e.g., a step-wise taper along a length of dilator 16), a different rate of taper at different parts of dilator 16, or both. By tapering in the distal direction, dilator 16 may serve to increase a diameter of the puncture made by needle 18 through the dermis into vasculature of the patient.

Dilator 16 also may extend into introducer sheath lumen 42, e.g., at distal introducer sheath end 40. For example, a proximal portion of dilator 16, proximal of exposed proximal dilator end 46, may taper or step down to a smaller outer diameter, e.g., an outer diameter that is substantially the same as (e.g., slightly smaller than) an inner diameter of introducer sheath lumen 42. In some examples, dilator 16 may extend the length of introducer sheath 14 through introducer sheath lumen 42 and hub 12 through hub lumen 34 to connect to proximal attachment 20. In other examples, dilator 16 may extend part of the length of introducer sheath 14 and/or hub 12 and be connected to proximal attachment 20 using an intermediary structure, such as one or more a wire, a ribbon, a tube, or the like.

Dilator 16 may be formed from any suitable material. Like introducer sheath body 36, dilator 16 may be formed from a biocompatible polymeric material. In some examples, the material from which dilator 16 is formed may be translucent or substantially transparent material, which may facilitate viewing of fluids within dilator lumen 48 through dilator 16. This may allow visualization of flashback of blood upon needle 18 cannulating vasculature of the patient. The biocompatible polymeric material may include, for example, a silicone, a fluoropolymer, a polyolefin, a fluorinated polyolefin, a polyurethane, a polyamide, a polyether, a polyether block amide, or the like. Dilator 16 may include a single layer or multiple layers. For example, dilator 16 may include a first layer that provides structural properties to dilator 16 and a lubricious coating on an external surface of the first layer. In some examples, dilator 16 includes a first layer including fluorinated ethylene propylene and a coating including a silicon, a first layer including a polyether block amide and a coating including a hydrogel, or the like.

Needle 18 is movably disposed in dilator lumen 48 and may extend from distal dilator end 46 out of dilator lumen 48. Needle 18 may define any suitable diameter (e.g., as measured on the Birmingham gauge scale). In some examples, needle 18 may be a micro puncture needle or an IV catheter needle. Needle 18 may define a needle lumen 50, which extends from a needle proximal end 52 to a needle distal end 54.

As best seen in FIG. 2, percutaneous vasculature access device 10 also may optionally include a guidewire 56. Guidewire 56 is configured to be positioned within introducer lumen 42 and dilator lumen 48 when in a retracted state. Guidewire 56 may be connected to guidewire actuator 24, e.g., by extending through introducer lumen 42 and hub lumen 34 to directly attach to guidewire actuator 24 or by being attached to an intermediary structure, such as one or more a wire, a ribbon, a tube, or the like, that is attached to guidewire actuator 24.

Guidewire 56 may define a diameter selected to allow advancing of guidewire 56 into needle lumen 50 and at least a distal portion of guidewire 56 out of needle distal end 54. As such, the diameter of guidewire 56 may depend on a gauge of needle 18.

Manipulators 20 include components that allow a user, such as a clinician or technician, to interact with percutaneous vasculature access device 10 to move components that are at least partially internal to a patient during the access procedure. Manipulators 20 may include needle cap 26, proximal attachment 22, and, optionally, guidewire actuator 24.

Proximal attachment 22 is removably attached to proximal hub end 30 of hub 12. For example, a distal end 62 of proximal attachment 22 may be friction fit to into proximal hub end 30, an outer surface of distal end 62 of proximal attachment 22 may include threads complementary to threads on an internal surface of hub 12, or the like.

Proximal attachment 22 extends from a proximal end 60 to a distal end 62. Proximal attachment 22 also defines a proximal attachment lumen 64, which extends from proximal end 60 to distal end 62. Proximal attachment lumen 64 is in communication with hub lumen 34, introducer sheath lumen 42, dilator lumen 48, and/or needle lumen 50. For example, proximal attachment lumen 64 may be substantially coaxial or coincident with hub lumen 34, introducer sheath lumen 42, dilator lumen 48, and/or needle lumen 50. As used herein, the terms “substantially coaxial” and “substantially coincident” mean “coaxial or coaxial to the extent permitted by manufacturing variances” and “coincident or coincident to the extent permitted by manufacturing variances,” respectively. Proximal attachment lumen 64 may be sized to allow passage of a needle hook 58, optionally, guidewire 56 (or an intermediary structure connecting guidewire 56 to guidewire actuator 24), and, optionally, guidewire actuator 24. In examples in which guidewire actuator 24 slides relative to proximal attachment 22 to move guidewire 56, a length of proximal attachment 22 may be at least great enough to allow guidewire actuator 24 to move sufficiently far to extend guidewire 56 to a desired distal-most position and retract guidewire 56 to a desired proximal-most position.

Proximal attachment 22 is directly attached to dilator 16 or an intermediary structure that is attached to dilator 16. In this way, proximal movement of proximal attachment 22 relative to hub 12 causes proximal movement of dilator 16 relative to introducer sheath 14. By removing proximal attachment 22 from proximal hub end 30, dilator 16 is moved proximally through introducer sheath lumen 42, through hub lumen 34, and out proximal end 30 of hub 12.

In some examples, proximal attachment 22 includes an optional slot 66 that extends axially along proximal attachment 22 (e.g., substantially parallel to a long axis of proximal attachment 22). Guidewire actuator 24 extends through slot 66 to attach to guidewire 56 or an intermediary structure configured to connect guidewire 56 to guidewire actuator 24. For example, as best seen in FIG. 3, guidewire actuator 24 may include a tab 68 extending from guidewire actuator body 70 radially inward through slot 66, where tab 68 is configured to attach to guidewire 56.

Slot 66 defines the limits of travel of guidewire actuator 24. For example, a proximal end 72 of slot 66 defines a proximal-most position of guidewire actuator 24, and, accordingly, a proximal-most retracted position of guidewire 56. Similarly, a distal end 74 of slot 66 defines a distal-most position of guidewire actuator 24, and, thus, a distal-most extended position of guidewire 56. In this way, the length of slot 66 may define a length of travel of guidewire actuator 24 in a direction parallel to the longitudinal axis of proximal attachment 22 (and device 10) relative to proximal attachment 22 and a length of travel of guidewire 56 in a direction parallel to the longitudinal axis of proximal attachment 22 relative to proximal attachment 22. In examples such as that shown in FIGS. 1-3, a movement of guidewire actuator 24 relative to proximal attachment 22 may cause a corresponding (e.g., 1:1) movement of guidewire 56.

In some examples, slot 66 includes one or more features that facilitate locking of guidewire actuator 24, e.g., maintaining guidewire actuator 24 at a selected longitudinal position. For example, as best seen in FIG. 1, slot 66 may include a side channel 76, into which guidewire actuator 24 may be slid, and which prevents proximal retraction of guidewire actuator 24 and guidewire 56. This may allow a user, such as a clinician or technician, to actuate guidewire actuator 24 into side channel 76 after fully distally extending guidewire 56, to prevent inadvertent distal retraction of guidewire 56 during subsequent manipulations.

In the examples illustrated herein, guidewire actuator 24 includes a body 70 that includes a ridged tab. The ridges may increase friction between body 70 and a finger or thumb of the user. In other examples, guidewire actuator 24 may include another type of manipulator configured to move guidewire 56 in a longitudinal direction, such as a free-spinning or ratcheting wheel, a rack and pinion, or the like.

In some examples, percutaneous vasculature access device 10 may omit guidewire 56. In examples in which percutaneous vasculature access device 10 omits guidewire 56, percutaneous vasculature access device 10 may also omit guidewire actuator 24, and proximal attachment 22 may omit slot 66 and side channel 76. In some such examples, proximal attachment 22 still attaches to dilator 16, either directly or via an intermediary structure. Hence, proximal attachment 22 may still be used to remove dilator 16 proximally through introducer sheath lumen 24, hub lumen 34, and out proximal end 30 of hub 12.

Manipulators 20 also include needle cap 26. Needle cap 26 is connected to needle 18 such that proximal movement of needle cap 26 relative to hub 12 (or proximal attachment 22) causes movement of needle 18 relative to hub 12 (or proximal attachment 22). For example, needle cap 26 may be connected to needle 18 by needle hook 58.

Needle hook 58 may include one or more wires, ribbons, coils, tubes, or the like, that are relatively rigid such that movement of proximal movement of needle cap 26 is translated into a corresponding proximal movement of needle 18 in a substantially 1:1 ratio. For example, needle hook 58 may be formed from a biocompatible metal, alloy, polymer, fabric, or the like.

As best seen in FIG. 2, in some examples, needle hook 58 may include a plurality of ribbons defining an aperture 78 between the plurality of ribbons. Aperture 78 may be fluidically coupled to needle lumen 50. The inclusion of aperture 78, in combination with the translucent or transparent material from which dilator 16 is formed, may allow a user of percutaneous vasculature access device 10 to visualize flashback of blood upon cannulating vasculature of a patient. In some examples, needle hook 58 may include a second aperture near needle cap 26, e.g., along a length of proximal attachment 22, or the same aperture 78 may extend the length of needle hook 58. The aperture along the length of proximal attachment 22 may allow guidewire actuator 24 to connect to guidewire 56, which is positioned radially inward from needle hook 58, as best seen in FIG. 2.

Needle cap 26 is configured to be removably attached to proximal end 60 of proximal attachment 22. For example, a needle cap 26 may be friction fit to into proximal end 60, an outer surface of a distal extension 80 of needle cap 26 may include threads complementary to threads on an internal surface of proximal attachment 22, or the like. In the examples of FIGS. 1-3, as best seen in FIG. 3, distal extension 80 of needle cap 26 is friction fit into proximal attachment lumen 64 of proximal attachment 22. Needle hook 58 is attached to distal extension 80. In other examples, needle hook 58 may be connected indirectly to distal extension 80 via an intermediary structure.

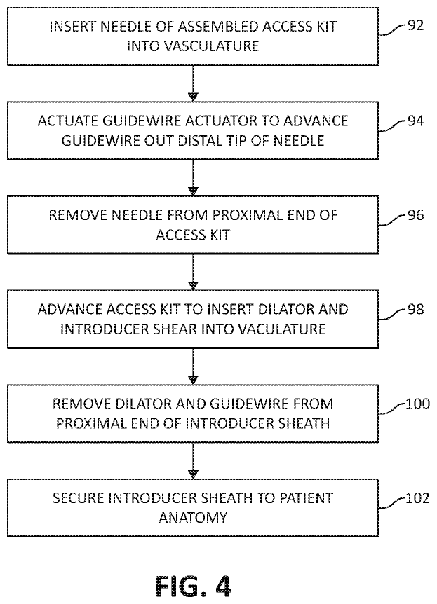

Percutaneous vasculature access device 10 may be used in a medical procedure to access vasculature of a patient, such as a radial, transradial, distal transradial, femoral, brachial, or carotid artery of a patient. FIG. 4 is flow diagram illustrating an example method for accessing vasculature using a percutaneous vasculature access device 10 of this disclosure. The technique of FIG. 4 will be described with respect to FIGS. 1-3 and 5-9, although it will be understood that percutaneous vasculature access device 10 of FIGS. 1-3 and 5-9 may be used to perform other techniques, and the technique of FIG. 4 may be used with other access devices.

The technique of FIG. 4 includes advancing percutaneous vasculature access device 10 relative to anatomy of a patient while percutaneous vasculature access device 10 is fully assembled to cause needle 18 of percutaneous vasculature access device 10 to be inserted percutaneously into vasculature of a patient (92). During the advancing, percutaneous vasculature access device 10 may be in the configuration shown in FIGS. 1 and 2, in which guidewire actuator 24 is positioned proximally in slot 66 such that a distal end of guidewire 56 is positioned proximal of needle proximal end 52. A user, such as a clinician or technician may grip percutaneous vasculature access device 10, e.g., at hub 12, and advance percutaneous vasculature access device 10 relative to anatomy of a patient to cause needle 18 of percutaneous vasculature access device 10 to be inserted percutaneously into vasculature of a patient. As dilator sheath 16 is translucent or visually transparent and aperture 78 of needle hook 58 is fluidically connected to needle lumen 50, the user may visualize blood flashback upon cannulating the vasculature. The user may stop advancing percutaneous vasculature access device 10 upon visualizing blood flashback.

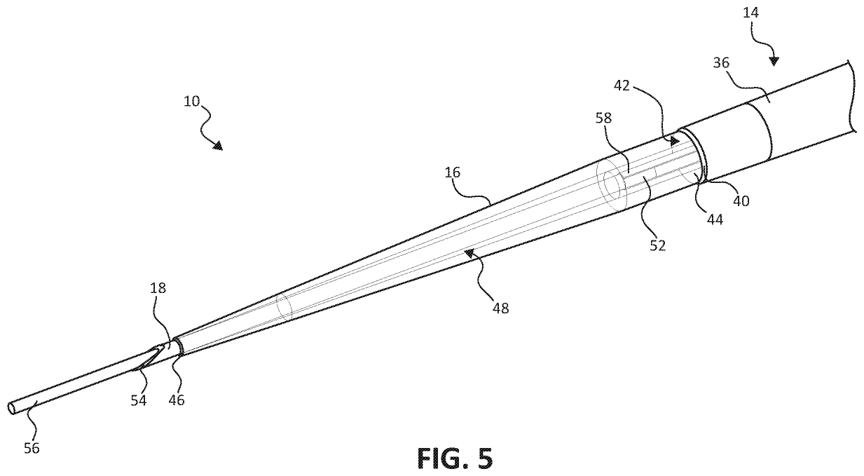

The user may then actuate guidewire actuator 24 to advance guidewire 56 out of needle distal end 54 via needle lumen 50 (94). For example, the user may slide guidewire actuator 24 distally along slot 66 until guidewire actuator 24 contacts the distal end 74 of slot 66, then slide guidewire actuator 24 circumferentially to position guidewire actuator 24 in side channel 76, as shown in FIG. 3. This results in guidewire 56 extending from needle distal end 54 as shown in FIG. 5. FIG. 5 is a perspective view illustrating a distal portion of percutaneous vasculature access device 10 after distal actuation of guidewire actuator 24, showing guidewire 56 advanced out of a distal end 54 of a needle lumen 50.

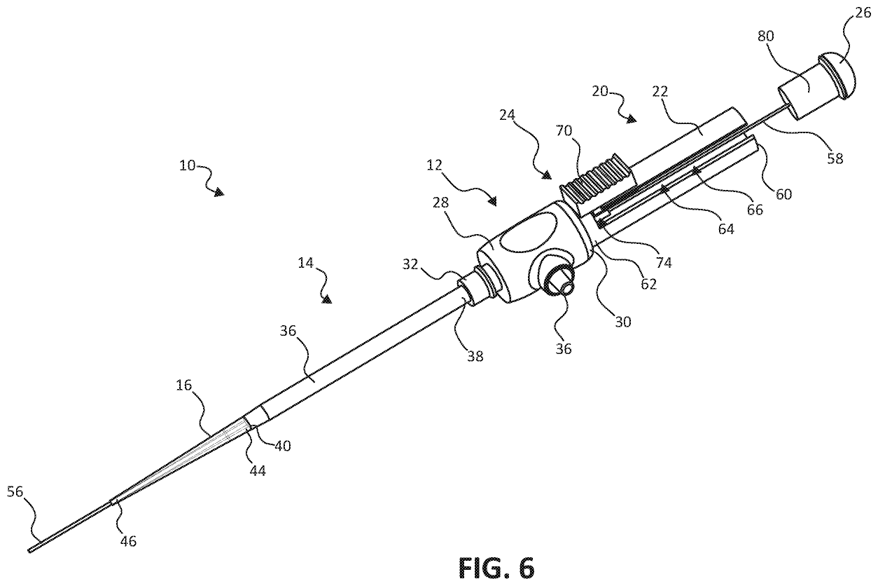

The user may then remove needle cap 26 from proximal end 60 of proximal attachment 22 to cause removal of needle 18 from the vasculature and out of proximal hub end 30 of hub lumen 34 (96). This procedure is shown in FIG. 6, which is a perspective view illustrating percutaneous vasculature access device 10 with needle cap 26 removed from a proximal end 60 of proximal attachment 22. As shown in FIG. 6, removal of needle cap 26 from proximal end 60 of proximal attachment 22 causes proximal movement of needle 18 through dilator lumen 48, introducer sheath lumen 42, and hub lumen 34, as needle cap 26 is connected to needle 18 using needle hook 58. Upon sufficient proximal movement of needle cap 26, needle 18 will proximally exit proximal hub end 30 of hub lumen 34 and proximal attachment lumen 64 at proximal end 60 of proximal attachment 22. This leaves dilator 16 directly adjacent to guidewire 56 with no needle 18 between dilator 16 and guidewire 56.

The user may then advance percutaneous vasculature access device 10 to advance guidewire 56 through the vasculature and advance dilator 16 and introducer sheath 14 into the vasculature (98). Guidewire 56 acts as a guide to facilitate navigation of the vasculature as dilator 16 and introducer sheath 14 are advanced into the vasculature. The taper of dilator 16 acts to increase a diameter of the puncture so introducer sheath 14 can enter the vasculature. In some examples, the user may disengage proximal attachment 22 from proximal hub end 30 prior to advancing percutaneous vasculature access device 10 (98) such that hub 12, introducer sheath 14 are movable independently of guidewire 56 and introducer sheath 14 may be advanced over guidewire 56.

Once introducer sheath 14 has been advanced the desired distance into the vasculature, the user may remove proximal attachment 22 from proximal hub end 30 to cause removal of guidewire 56 and dilator 16 from the vasculature and out of proximal hub end 60 of hub lumen 34 (100). As described above, dilator 16 may be connected to proximal attachment 22, either directly or via an intermediary structure. Guidewire 56 may be connected to guidewire actuator 24, either directly or via an intermediary structure. By disengaging distal end 62 of proximal attachment 22 from proximal hub end 60 and moving proximal attachment 22 proximally, dilator 16 and guidewire 56 may be moved proximally through introducer sheath lumen 42, hub lumen 34, and out proximal hub end 30. This leaves introducer sheath 14 in the vasculature of the patient, attached to hub 12, as shown in FIG. 7. FIG. 7 is a perspective view illustrating hub 12 and introducer sheath 14 of percutaneous vasculature access device 10, after removal of proximal attachment 22, dilator 16, guidewire 56, needle 18, and needle cap 26. Introducer sheath 14 and hub 12 then may be used in a medical procedure to introduce a medial device or medical supply into the vasculature of the patient. Although not shown in FIG. 7, in some examples, percutaneous vasculature access device 10 also may include a sheath cap on hub 12.

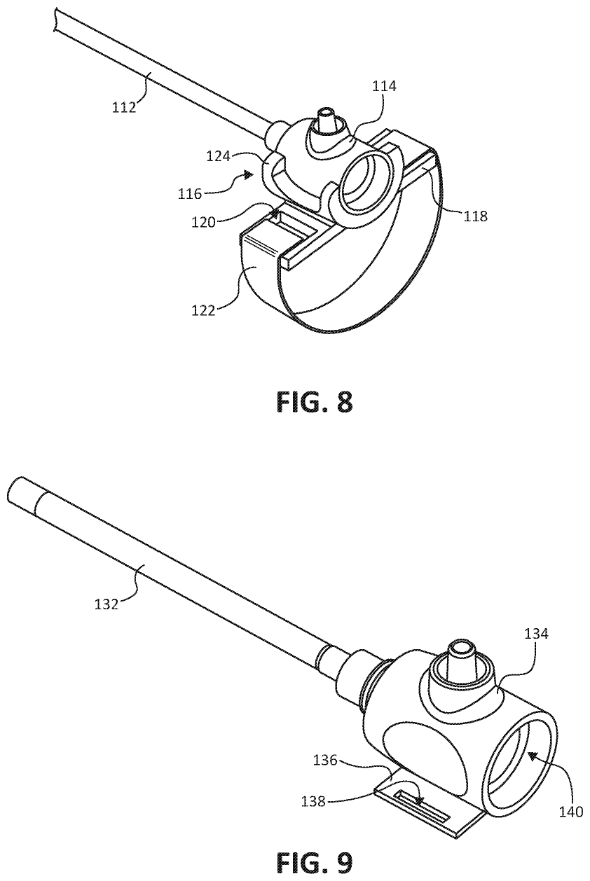

In some examples, the user may optionally secure hub 12 to an anatomical structure of the patient using a hub clip band (102). For example, retrograde blood flow may exert a force on introducer sheath 14 encouraging introducer sheath 14 out of the vasculature. This may be particularly problematic for introducer sheath 14 that are shorter or include a hydrophilic coating, although similar issues may arise with any introducer sheath 14. FIG. 8 is a perspective view of an example introducer sheath 112 and an example hub 114 engaged with an example hub band clip 116. Introducer sheath 112 and hub 114 may be similar to or substantially the same as introducer sheath 14 and hub 12 of FIGS. 1-3 and 5-7, aside from the differences described herein. Hub band clip 116 includes a pair of extensions 118 that each includes a respective slot 120 configured to receive a respective end of a band 122. Band 122 may be sized to fit around an anatomical structure of a patient (e.g., a leg, arm, wrist, hand, or the like) and may be elastic to exert pressure on the anatomical structure and secure hub band clip 116 to the anatomical structure.

Hub band clip 116 also includes a plurality of hub engagement features 124 that are sized and shaped to engage with complementary clip features of hub 114 to retain hub 114. For example, hug 114 may include a shape and size that defines one or more clip features, and that are configured to engage with hub engagement features 124 of hub band clip 116 to retain hub 114 in hub band clip 116. In this way, hub band clip 116 may secure hub 114 and introducer sheath 112 relative to patient anatomy.

FIG. 9 illustrates another way to secure an introducer sheath and hub relative to patient anatomy. FIG. 9 is a perspective view of an example introducer sheath 132 and an example hub 134 that includes side attachments 136 configured to receive a band or tape 138. Introducer sheath 132 and hub 134 may be similar to or substantially the same as introducer sheath 14 and hub 12 of FIGS. 1-3 and 5-7, aside from the differences described herein. Although a single side attachment is shown in FIG. 9, the other side of hub 13 includes a similar side attachment. Each of side attachments 136 includes a respective slot 138 for receiving a respective end of the band or tape. FIG. 9 also shows hub lumen 140.

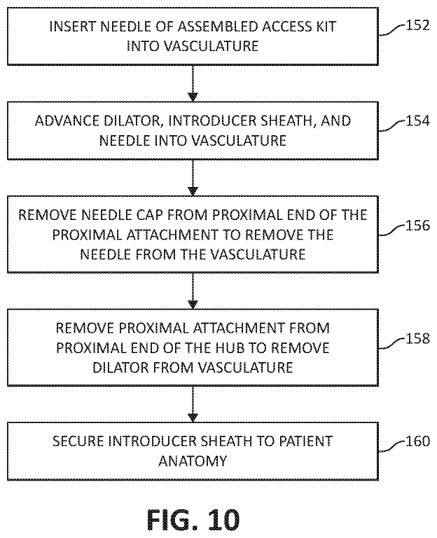

As described above, in some examples, a percutaneous vasculature access device may omit a guidewire and guidewire actuator. FIG. 10 is a flow diagram illustrating an example method for accessing vasculature using a percutaneous vasculature access device of this disclosure that omit a guidewire and guidewire actuator. The technique of FIG. 10 will be described with respect to FIGS. 1-3 and 5-9, although it will be understood that percutaneous vasculature access device 10 of FIGS. 1-3 and 5-9 may be used to perform other techniques, and the technique of FIG. 10 may be used with other access devices.

The technique of FIG. 10 includes advancing percutaneous vasculature access device 10 relative to anatomy of a patient to cause needle 18 of percutaneous vasculature access device 10 to be inserted percutaneously into vasculature of a patient (152). During the advancing, percutaneous vasculature access device 10 may be in the configuration shown in FIGS. 1 and 2, in which guidewire actuator 24 is positioned proximally in slot 66 such that a distal end of guidewire 56 is positioned proximal of needle proximal end 52. A user, such as a clinician or technician may grip percutaneous vasculature access device 10, e.g., at hub 12, and advance percutaneous vasculature access device 10 relative to anatomy of a patient to cause needle 18 of percutaneous vasculature access device 10 to be inserted percutaneously into vasculature of a patient. As dilator sheath 16 is translucent or visually transparent and aperture 78 of needle hook 58 is fluidically connected to needle lumen 50, the user may visualize blood flashback upon cannulating the vasculature. The user may stop advancing percutaneous vasculature access device 10 upon visualizing blood flashback.

The user may then continue to advance percutaneous vasculature access device 10 to advance needle 18, dilator 16, and introducer sheath 14, thus advancing dilator 16 and introducer sheath 14 into the vasculature (154). The taper of dilator 16 acts to increase a diameter of the puncture so introducer sheath 14 can enter the vasculature.

The user may then remove needle cap 26 from proximal end 60 of proximal attachment 22 to cause removal of needle 18 from the vasculature and out of proximal hub end 30 of hub lumen 34 (156). In some examples, rather than removing needle cap 26 after advancing dilator 16 and introducer sheath 14 into the vasculature (154), the user may remove needle cap 26 (156) before advancing dilator 16 and introducer sheath 14 into the vasculature (154).

Once introducer sheath 14 has been advanced the desired distance into the vasculature, the user may remove proximal attachment 22 from proximal hub end 30 to cause removal of dilator 16 from the vasculature and out of proximal hub end 60 of hub lumen 34 (158). As described above, dilator 16 may be connected to proximal attachment 22, either directly or via an intermediary structure. By disengaging distal end 62 of proximal attachment 22 from proximal hub end 60 and moving proximal attachment 22 proximally, dilator 16 may be moved proximally through introducer sheath lumen 42, hub lumen 34, and out proximal hub end 30. This leaves introducer sheath 14 in the vasculature of the patient, attached to hub 12, as shown in FIG. 7.

In some examples, the user may optionally secure hub 12 to an anatomical structure of the patient using a hub clip band (160), e.g., using one of the devices shown in FIG. 8 or 9.

Various examples have been described. Any combination of the described systems, devices, operations, or functions is contemplated. These and other examples are within the scope of the following claims.

What is claimed is:

1. A percutaneous vasculature access device comprising:
a hub defining a proximal hub end, a distal hub end, and a hub lumen extending from the proximal hub end to the distal hub end;
an introducer sheath defining a proximal introducer sheath end, a distal introducer sheath end, and an introducer sheath lumen extending from the proximal introducer sheath end to the distal introducer sheath end, wherein the proximal introducer sheath end is configured to be connected to the distal hub end, and wherein the introducer sheath lumen is in communication with the hub lumen;
a dilator defining a dilator lumen;
a proximal attachment configured to be connected to the dilator, wherein the proximal attachment is configured to be removably connected to the proximal hub end, and wherein the dilator is configured to extend distally from the distal introducer sheath end when the proximal attachment is configured to be removably connected to the proximal hub end;
a needle configured to be movably disposed within the dilator lumen; and
a needle cap configured to be removably connected to a proximal end of the proximal attachment, wherein the needle cap is configured to be connected to the needle such that movement of the needle cap causes movement of the needle.

2. The percutaneous vasculature access device of claim 1, wherein the needle defines a needle lumen, the device further comprising a guidewire configured to be advanced into the needle lumen.

3. The percutaneous vasculature access device of claim 2, further comprising a guidewire actuator configured to be connected to the guidewire and configured to cause the guidewire to move longitudinally relative to the needle.

4. The percutaneous vasculature access device of claim 2, wherein the proximal attachment defines a longitudinal slot, the device further comprising a guidewire actuator configured to be connected to the guidewire and configured to move longitudinally in the longitudinal slot relative to the proximal attachment to move the guidewire relative to the needle.

5. The percutaneous vasculature access device of claim 3, wherein the guidewire actuator is configured to be connected to the guidewire so that movement of the guidewire actuator causes a corresponding movement of the guidewire.

6. The percutaneous vasculature access device of claim 3, wherein the guidewire actuator comprises a ridged tab.

7. The percutaneous vasculature access device of claim 2, wherein proximal removal of the proximal attachment from the hub proximal end is configured to cause proximal removal of the guidewire and the dilator out of the hub lumen.

8. The percutaneous vasculature access device of claim 1, wherein the dilator is visually translucent or visually transparent.

9. The percutaneous vasculature access device of claim 1, further comprising a needle hook configured to connect the needle to the needle cap.

10. The percutaneous vasculature access device of claim 9, wherein the needle hook comprises at least one aperture fluidically coupled to the needle lumen.

11. The percutaneous vasculature access device of claim 10, wherein the proximal attachment defines a proximal attachment lumen extending from the proximal end of the proximal attachment to a distal end of the proximal attachment, and wherein the needle hook extends from the needle to the needle cap through the proximal attachment lumen.

12. The percutaneous vasculature access device of claim 1, wherein proximal removal of the needle cap is configured to cause proximal removal of the needle out of the hub lumen.

13. The percutaneous vasculature access device of claim 1, wherein the hub further comprises side attachments configured to receive a band or tape to secure the hub and the introducer sheath to a patient.

14. The percutaneous vasculature access device of claim 1, wherein the hub further comprises at least one clip feature configured to engage with a hub band clip, wherein the hub band clip is configured to secure the hub and the introducer sheath to a patient.

15. The percutaneous vasculature access device of claim 1, wherein the hub further comprises a side access port.

16. The percutaneous vasculature access device of claim 1, wherein proximal removal of the proximal attachment from the hub proximal end is configured to be proximal removal of the dilator out of the hub lumen.

17. A percutaneous vasculature access device comprising:
a hub defining a proximal hub end, a distal hub end, and a hub lumen extending from the proximal hub end to the distal hub end;
an introducer sheath extending from the distal hub end, wherein the introducer sheath defines a proximal introducer sheath end, a distal introducer sheath end, and an introducer sheath lumen extending from the proximal introducer sheath end to the distal introducer sheath end, wherein the proximal introducer sheath end is configured to be connected to the distal hub end, and wherein the introducer sheath lumen is in communication with the hub lumen;
a dilator defining a dilator lumen;
a proximal attachment configured to be connected to the dilator, wherein the proximal attachment is configured to be removably connected to the proximal hub end, wherein the dilator is configured to extend distally from the distal introducer sheath end when the proximal attachment is configured to be removably connected to the proximal hub end;
a needle configured to be movably disposed within dilator lumen, wherein the needed defines a needle lumen;
a needle cap configured to be removably connected to a proximal end of the proximal attachment, wherein the needle cap is configured to be connected to the needle such that movement of the needle cap causes movement of the needle, wherein proximal removal of the needle cap from the proximal attachment is configured to cause proximal removal of the needle out of the hub lumen;
a guidewire configured to be movably disposed in the needle lumen; and
a guidewire actuator configured to be connected to the guidewire, wherein movement of the guidewire actuator is configured to cause movement of the guidewire relative to the needle, and wherein proximal removal of the proximal attachment from the hub proximal end is configured to cause proximal removal of the dilator and the guidewire out of the hub lumen.

18. The percutaneous vasculature access device of claim 17, wherein the proximal attachment defines a longitudinal slot, wherein the guidewire actuator is configured to move longitudinally in the longitudinal slot relative to the proximal attachment to move the guidewire longitudinally relative to the needle.

19. The percutaneous vasculature access device of claim 17, wherein the guidewire actuator is configured to connected to the guidewire so that movement of the guidewire actuator causes a corresponding movement of the guidewire.

20. The percutaneous vasculature access device of claim 17, wherein the guidewire actuator comprises a thumb tab.

21. The percutaneous vasculature access device of claim 17, wherein the dilator is visually translucent or visually transparent.

22. The percutaneous vasculature access device of claim 17, further comprising a needle hook configured to connect the needle to the needle cap.

23. The percutaneous vasculature access device of claim 22, wherein the needle hook comprises at least one aperture fluidically coupled to the needle lumen.

24. The percutaneous vasculature access device of claim 23, wherein the proximal attachment defines a proximal attachment lumen extending from the proximal end of the proximal attachment to a distal end of the proximal attachment, and wherein the needle hook is configured to extend from the needle to the needle cap through the proximal attachment lumen.

25. The percutaneous vasculature access device of claim 17, wherein the hub further comprises side attachments configured to receive a band or tape configured to secure the hub and the introducer sheath to a patient.

26. The percutaneous vasculature access device of claim 17, wherein the hub further comprises at least one clip feature configured to engage with a hub band clip, wherein the hub band clip is configured to secure the hub and the introducer sheath to a patient.

27. The percutaneous vasculature access device of claim 17, wherein the hub further comprises a side access port.
